(19) United States
(12) Patent Application Publication
(10) Pub. No.: US 2007/0095699 A1
(43) Pub. Date: May 3, 2007

(54) Armoured sterilization case tray with internal wrap/filter

(75) Inventors: Allan S. Frieze, Alpine, NJ (US); Marcia A. Frieze, Alpine, NJ (US)

Correspondence Address: Irving M. Fishman, Suite 1422, North Tower, 89 Headquarters Plaza, Morristown, NJ, 07960, US

(73) Assignee: Case Medical, Inc.

(21) Appl. No.: 11/259,764

(22) Filed: Oct. 27, 2005

(51) Int. Cl. A61B 19/02

(52) U.S. Cl. 206/438

(57) ABSTRACT

A sterilization container system for sterilizing objects therein comprising (a) an outer case having a bottom and four outer case side walls, said outer case bottom and said outer case four side walls defining an outer case interior; (b) an outer case lid; (c) a liner; (d) an inner tray having an inner tray bottom and four inner tray side walls, said inner tray bottom and four inner tray side walls defining an inner tray interior; and (e) optionally, an inner tray lid; wherein in use, said inner tray carries one or more objects for sterilization in said inner tray interior, said inner tray optionally having said inner tray lid thereon and wrapped within said liner to form a wrapped inner tray, and said wrapped inner tray located in said outer case interior, with said outer case lid closing said interior of said outer case.

### CROSS-REFERENCE TO RELATED APPLICATIONS

Not applicable.

### STATEMENT REGARDING FEDERALLY SPONSORED RESEARCH OR DEVELOPMENT

Not Applicable

### FIELD OF THE INVENTION

The present invention relates to sterilization container trays, especially trays for disinfection and sterilization of medical instruments.

### BACKGROUND OF THE INVENTION

Rigid, reusable, sealed sterilization containers are known in the art as evidenced from U.S. Ser. No. 10/295,758; U.S. Ser. No. 10/070,621; U.S. Pat. No. 6,468,482; U.S. Pat. No. 6,589,477; U.S. Pat. No. 5,968,459; for example, all of which are incorporated herein in their entirety. These rigid, reusable, sealed containers are costly to manufacture and are therefore offered commercially in set sizes. At times, a device that requires sterilization and packaging does not fit the standard sized containers. Specialty sized rigid, reusable, sealed containers become prohibitively expensive and when not in use require excessive storage space.

As an alternative to the use of these expensive and space consuming specialty rigid reusable cases, placing the device or instrument into a tray and then wrapping the product in a disposable medical grade wrapper or muslin or other reusable wrap has been tried. Other alternatives that have been tried include using a peel pouch or a disposable bag. The devices to be sterilized are usually wrapped in double layers to avoid strike through and tears in the wrapping. FIGS. 1 and 2 demonstrate two typical wrapping techniques of this type. However, it has been noted that about 10% and possibly more of all instrument sets that are wrapped in such fashions require reprocessing due to torn or punctured wrappers, may result in wet packs, or have handling problems (since wrapped trays do not have handles for lifting and carrying), and the unwrapped trays are awkward to wrap and fasten.

Thus, there is a substantial unmet need in having an alternative to both the rigid, reusable, sealed container and to the commonly used wrapped tray with its associated handling and sterility maintenance issues.

### OBJECTS OF THE INVENTION

It is therefore an object of the invention to provide a sterilization wrapping configuration that is readily adaptable to varied sized devices in need of sterilization.

It is another object of the invention to provide a sterilization wrapping configuration that has improved resistance to being pierced by the instruments being wrapped and sterilized.

Yet another object of the invention is to provide a sterilization tray that is rigid and can be easily wrapped.

Still further objects of the present invention will be appreciated by those of ordinary skill in the art.

### BRIEF SUMMARY OF THE INVENTION

These and other objects of the invention are surprisingly achieved by a system utilizing (1) a perforated outer sterilization container, (2) a sterilant permeable, generally contaminant impermeable liner, (3) a perforated inner tray and (4) a lid, wherein the outer container and the inner tray are rigid, reusable members and the impermeable liner is located between the outer container and the inner tray. The liner extends sufficiently that after placing devices for sterilization into the tray, the liner can be folded over the rims of the inner tray to create a wrapped tray containing such instruments, and the lid is placed thereover to secure the wrapped tray in place. The closed unit can then be sterilized in conventional manners as desired, such as steam, gas, or plasma sterilization.

### BRIEF DESCRIPTION OF THE DRAWINGS

FIG. 1 depicts known square fold double wrapping.

FIG. 2 is depicts an alternative double wrapping technique.

FIG. 3 is a picture of an embodiment of the present invention in which the inner tray has already been wrapped.

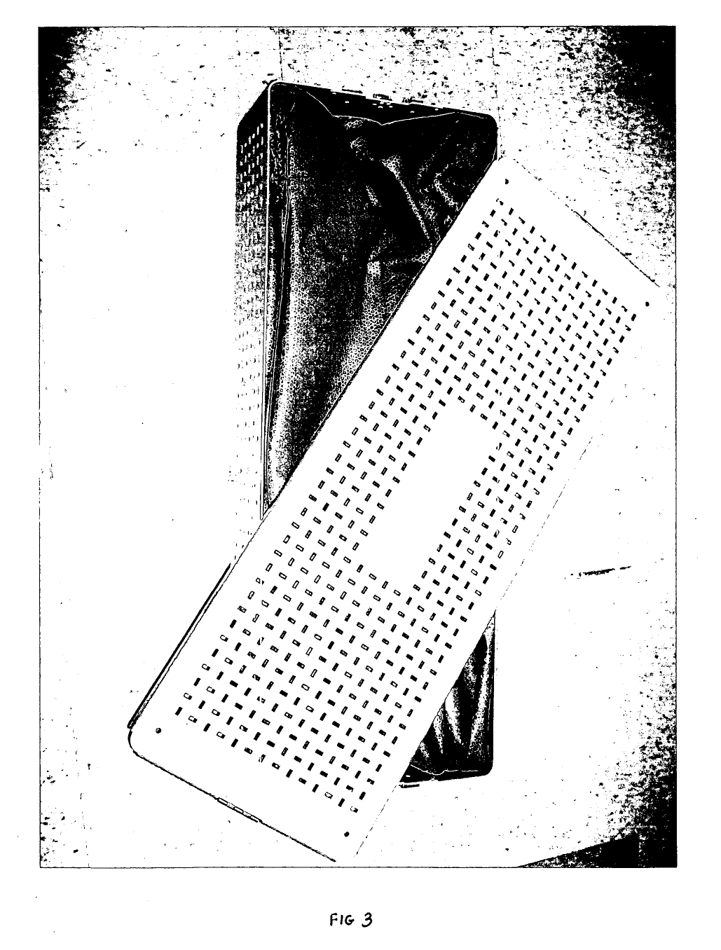

FIG. 4 is an exploded view of one embodiment of the invention before wrapping of the inner tray.

FIG. 5 is a view of a hinged outer case for use in the present invention.

FIG. 6 is a view of a hinged case for use in the present invention wherein the outer lid is hinged to the rest of the case.

### DETAILED DESCRIPTION OF THE INVENTION

The present invention is directed to a sterilization tray and system for disinfection of instruments and devices, most typically (but not limited to) medical, dental, laboratory, and veterinary instruments or devices requiring disinfection.

In its simplest form, the present invention requires an outer casing (1), a liner (2), an inner tray (3), and an outer casing lid (4). When in use, the liner (2) will be located between the outer casing (1) and the inner tray (3), and will of sufficient size that once the inner tray (3) is loaded with the object to be sterilized, the ends of the liner can be suitably folded over the rim of the inner tray (3) to enclose the inner tray (3) and its contents. The outer casing lid (4) is used to enclose the wrapped inner tray (3) within the outer casing (1). At least a portion of the outer casing (1) and outer casing lid (4) are permeable and preferably at least a portion of the outer casing (1) and outer casing lid (4) is perforated. The outer casing (1) may be perforated and the outer casing lid (4) may be solid; the outer casing lid (4) may be perforated and the sides or base of the outer case (1) may be solid, or both the outer case (1) and the outer case lid (4) may be perforated. In a preferred embodiment, at least one of the surfaces of the outer case (1) is perforated (i.e., one or more of the bottom and side walls of the outer case (1) is perforated); and in a highly preferred embodiment, all of the surfaces of the outer casing (1) (exclusive of edges) are perforated. The perforations on any particular surface of the outer case (1) or the outer case lid (4) may be in discrete patterns or randomly or regularly placed. A label may be added to an exterior surface of the outer case (1) (but preferably not the bottom) or the outer case lid (4), preferably each. In general there may be a portion of the outer case lid (4) or of one or more surfaces of the outer case (1) that is not perforated that allows for easy labeling and handling of a particular outer case (1) when closed with the outer case lid (4) in place. In a desirable embodiment, a portion of at least one of the outer case side walls and/or the outer case lid (4) is not perforated for the purpose of affixing a label thereto. This portion may be in any convenient location on the surface of such side wall and/or the outer case lid (4), but advantageously it is centrally located.

The outer case (1), outer case lid (4), and inner tray (3) may be of any suitable material for a sterilization container that is compatible with the sterilization mode. Thus, an outer case (1), outer case lid (4), and inner tray (3) that are intended for use in steam sterilization may be made of any materials that are compatible with steam sterilization and still result in a rigid container. As such, materials such as metals, plastics, composites and hybrids thereof are suitable. The outer case, outer case lid, and inner tray may be of any suitable material for a sterilization container intended for use in gas. For outer case (1), outer case lid (4), and inner tray (3) that are intended for use in gas plasma sterilization, these components can be fabricated from materials that are compatible with gas plasma sterilization, such as metal, anodized metal, conductive plastics, etc., composites thereof and hybrids thereof. Where gas plasma sterilization is used and the outer case lid (4) and outer case (1) are one or more of these materials, the inner tray (3) may also be one of these materials, but may also be constructed of other material that is not detrimental to the system as a whole. As such, in addition to metal, anodized metal, autoclavable or conductive plastics, etc., composites thereof, and hybrids thereof, the inner tray (3) may be made of suitable metal, plastic, hybrid or composite materials, or the like.

Suitable materials for the construction of the outer case (1), outer case lid (4), and inner tray (3) include, without limitation, metals such as stainless steel, aluminum, titanium, and magnesium, as well as alloys of any of these, plastics (which in the case of gas plasma suitable products are conductive plastics), metal filled plastics, carbon filled plastics, carbon/plastic composites and hybrids, metal/plastic composites and hybrids, and the like. Each of the metals or metal alloys may or may not have an anodic coating thereon, but preferably do have the anodic coating, especially if the metal is sensitive to the sterilant in question. A highly preferred metal is aluminum or aluminum alloy, especially aluminum alloy 0019, any of the series 2000 alloys, any of the series 5000 alloys, or any of the series 6000 alloys. A particularly suitable one of the series 5000 is 5052 and a particularly suitable one of the series 6000 is 6061. The anodic coating, when present in devices that are used in sterilization modes other than gas plasma sterilization, may be of any desired thickness. For devices that will be used in gas plasma sterilization, the anodic thickness is preferably maintained at not greater than about 0.5 mills (0.0005 inches), more preferably not greater than about 0.4 mils (0.0004 inches), more preferably not greater than about 0.35 mils (0.00035 inches), and while any such thickness below the maximums indicated here are suitable, in highly preferred embodiments, the anodic thickness is at least about 0.05 mils (0.00005 inches), more preferably at least about 0.1 mils (0.0001 inches), even more preferably at least about 0.15 mils (0.00015 inches), still more preferably at least about 0.2 mils (0.0002 inches), and most preferably at least about 0.25 mils (0.00025 inches). When the metal used in any of the above components is aluminum or an aluminum alloy, the metal or alloy may be optionally heat treated, if desired, preferably to T4, T5, or T6, with T4 or T5 being preferably as they are easier to attain in practice. However, heat treatment of the metal is not required. Still, heat treatment is not a requirement of the invention.

When plastics are used for any of the outer case (1), the inner tray (3), and/or the outer case lid (4), the plastic is any autoclavable plastic, with polypropylene, polysulfone, and liquid crystal polymer (LCP) being preferred. When a conductive component is necessary, but the foregoing materials are not themselves conductive, such as polypropylene, etc., a composite or hydbrid material such as a metal or carbon filled plastic or a metal or carbon composite with the non-conductive material can be used. Metal or carbon that are completely or nearly completely contained within the plastic (i.e. will not be significantly exposed directly to the sterilant can be chosen from any metal or carbon source that will provide the requisite electronic conductivity, without concern for sensitivity to the sterilant). Where the metal or other material is used in a fashion that it will be exposed to the sterilant, it is preferable to utilize such materials that are resistant to corrosion by the sterilant or to apply protective layers such as anodic coatings on metals. As an alternative to this, a conductive surface may be applied in any suitable manner to a non-conductive surface. As such, an aluminum layer may be applied to a non-conductive frame in any convenient manner, such as by wrapping the surfaces with metal foil or by vapor deposition and other techniques known in the art. Once the conductive layer has been applied and the perforation made, any anodic layer that is deemed needed should then be applied so that the entire exposed conductive surface can be layered with an anodic coating. While applying the anodic coating earlier is possible, doing so allows for small segments of the conductive surface to be exposed to sterilant when in use and therefore leads to early product failure.

The outer case (1), outer case lid (4), and inner tray (3) (when constructed of metal) can be constructed from sheet metal that has been pre-perforated in the desired manner, and is then bent into shape and if desired then anodized. Anodization before perforation and bending into shape is also suitable. Conductive plastics do not require the anodization layer and may be cast directly into the shape desired.

The outer case (1) may be formed with protrusions (7) or indentations (not shown) that mate with indentations (6) or protrusions (not shown), respectively, in outer case lids (4) so that one outer case (1) is readily and stably stackable on top of the outer case lid (4) acting as a closure for another outer case (1). These protrusions and indentations are most suitably spaced so that when engaged there is a clearance space between one outer case bottom (5) of one unit and the outer case lid (4) of another unit below it. The protrusions (7) on the outer case bottoms (5) may be sufficiently large as to be more recognizable as legs and feet and may be permanently attached to the case, formed as a unitary piece when the outer case is bent into shape, or may be attached to the case in a permanent or removable manner. When the legs and feet are attached separately to the outer case (by either permanent or removable means), they may be constructed of any suitable materials that are compatible with the sterilization method for which they are intended. They are not required to be of the same materials as the outer case, lid and/or inner tray, but preferably they are constructed from a material selected from the same group of materials that are suitable for the outer case. Most preferably, such legs and feet are made of the same material as the outer case.

As previously stated, the perforations (8)(9) present in the outer case (1) surfaces, the outer case lid (4) surfaces, and the inner tray (3) surfaces may be in any desired pattern or placed randomly. Most preferably, the pattern and sizing of the perforations (9) in the inner tray (3) are such that when the inner tray (3) is placed on the liner (2) and in the outer case (1), the perforations (9) in the inner tray (3) are offset from the perforations (8) in the outer case (1) so as to prevent any sharp portions of the instruments or devices being sterilized from being able to strike through from the inner tray (3) through the liner (2) and through the outer case (1). Any sharp points which might penetrate the perforations (9) of the inner tray would then be prevented from piercing the liner (2) as it would strike a solid surface of the outer case (1). The offset of the perforations also aids in creating a tortuous path for the sterilant to follow and thereby aid in dispersing the sterilant to all surfaces of the object being sterilized. The inner tray (3) may also have an engageable inner tray lid (10) that is similar to the lid for the outer case (1), except that it is sized for the inner tray (3). While the inner tray lid (10) is not absolutely required, it does provide a flat surface for use in the folding of the liner (2) when wrapping the inner tray (3). The inner tray lid (10) is particularly useful if one wishes to place the objects to be sterilized into the inner tray (3), wrap the inner tray (3) with the liner (2) and then place the wrapped inner tray into the outer case (1), as the inner tray lid (10) provides a rigid top surface against which the liner (2) can be pressed during the wrapping operation, secured by tape or a fastener without breaking the liner (2), and provides firm surfaces for transport of the wrapped inner tray to the outer case (1).

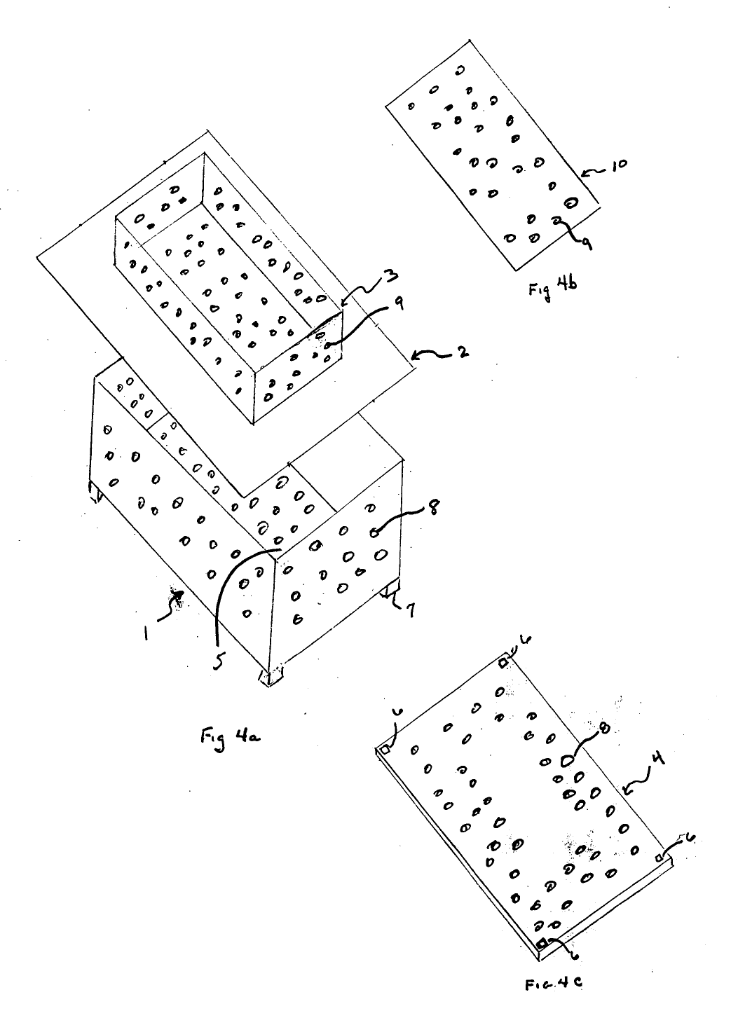

The liner (2), which is placed between the outer case (1) interior surface and the inner tray (3) outer surface, serves to wrap the inner tray (3) and the objects to be sterilized that are contained therein. The liner also advantageously serves as a microbial filter so that the once sterilized, the wrapped inner tray will remain sterile until use. The liner (2) material is thus selected from the group consisting of disposable or reusable materials such as medical grade cellulosic materials (for example paper, etc.); polypropylene or other non-woven polyolefins; linen or muslin, etc; or synthetic wrap (for example Gortex, Teflon, polyfoil compounds, tyvek, etc.); or mixtures or blends. In addition, the liner (2) may be a composite of multiple layers where each layer provides some, but not all of the desired characteristics; however, since multiple layer liners become increasingly difficult to properly fold, their use is less advantageous, but still within the scope of the present invention. The liner (2) material may be configured in flat sheets (as shown in FIG. 4), in peel pouches, or bags. Especially useful are bags and pouches with self closures contained therein. Flat sheets are also a preferred configuration as it allows for use of one size sheet for multiple sizes of inner trays (3) to be wrapped. Furthermore, as the outer case (1) provides a secondary protective surface for the contents, the liner (2) itself may be a single wrap as opposed to double wrapping or sequential layering of multiple wrap layers.

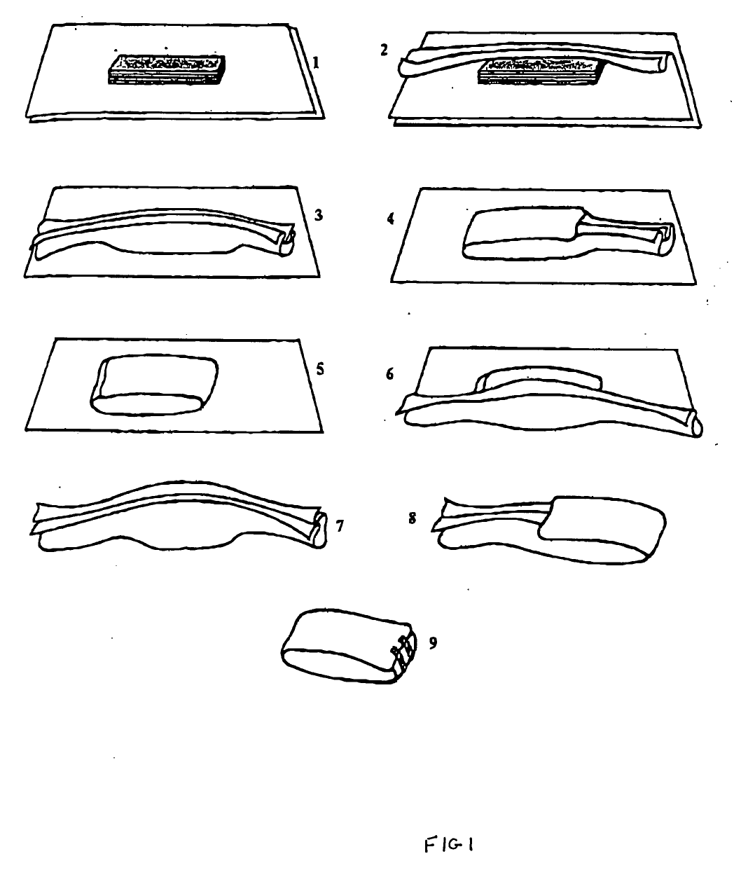

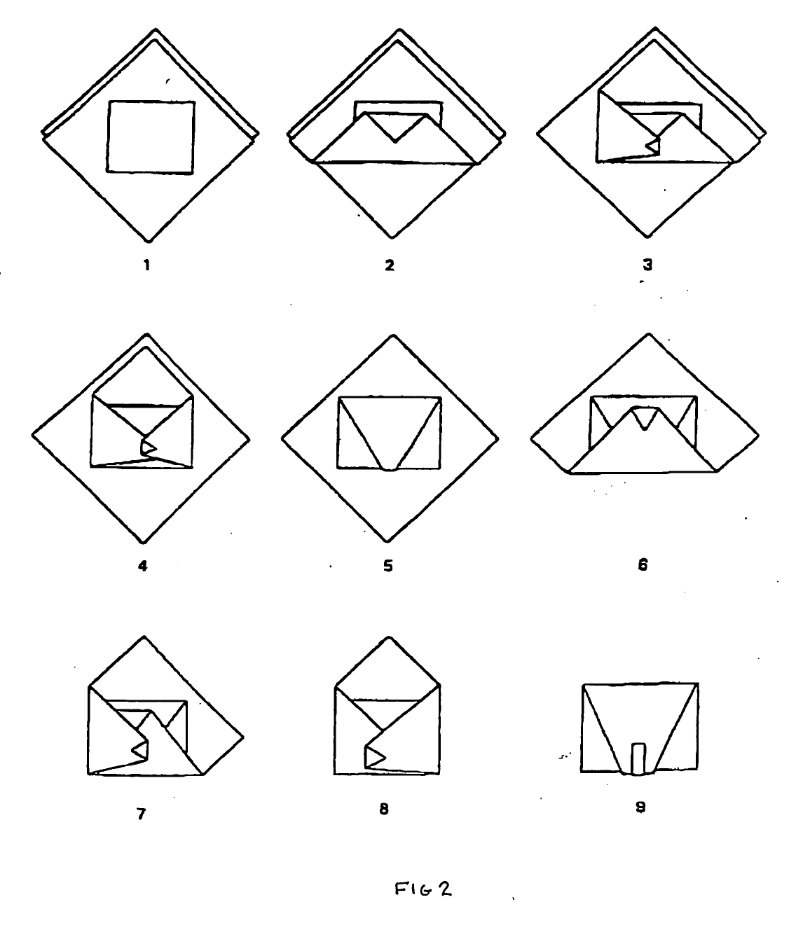

The liner (2) may be folded around the inner tray to enclose it (and the objects to be sterilized) in any manner conveniently known in the art. Two such methods are shown for double wrapping in FIGS. 1 and 2. Single wrapping in the same manner is perfectly suitable for the present invention. Still other wrapping methods will be apparent to those of ordinary skill in the art. Once wrapped, the folded liner (2) is fastened with any suitable fastening manner, such as adhesive, tape, clips, clamps, etc. In the case of a reusable liner (2) material, a particularly suitable fastening mechanism is the use of Velcro, which would be suitably attached to the liner (2) material when the liners (2) are made. Where the liner (2) material is configured as a bag or pouch, self closures as typically seen in plastic sandwich and freezer bags are also suitable.

The outer case (1) and/or the outer case lid (4) may be equipped with one or more latching means (not shown) to secure the outer case (1) and the outer case lid (4) together. Optional clips or holders located externally to the outer case (1) and/or outer case lid (4) may be present for attachment of a tamper-proof seal, and are preferably employed. Any tamper-proof or tamper-resistant sealing mechanism known in the art that is compatible with the sterilization procedure is suitable for use in the present invention, and those of ordinary skill will be well aware of such methods.

The inner surfaces of the outer case (1) and the outer surfaces of the inner tray (3) are preferably smooth, and free from rough edges at the perforation (8)(9) hole edges so as to minimize snagging of the liner (2) (and potentially causing a tear in the liner (2)) in the process of placing the liner (2) and inner tray (3) into the outer case (1). The inner tray (3) protects the liner (2) from potential tears from the object being sterilized, while the outer case (1) protects the liner (2) from potential tears and compromised sterility maintenance from the point the inner tray (3) is placed inside the outer case (1), through the sterilization procedure, through storage, until use.

The outer case is preferably equipped with one or more handles (not shown) for ease of handling the assembled sterilization unit. These handles may be in a fixed position which does not interfere with stacking of various units, or may be in any position if they are movable and can be moved to a position that allows for stacking of individual units. Such handles are well known in the art. Handles may be made of any suitable material and may, but need not, be made from the same materials as the rest of the outer case. Handles may be permanently affixed to the outer case (1) or may be removable by engagement and disengagement of handle attaching means (not shown in the figures) located at any suitable position of the outer case (1).

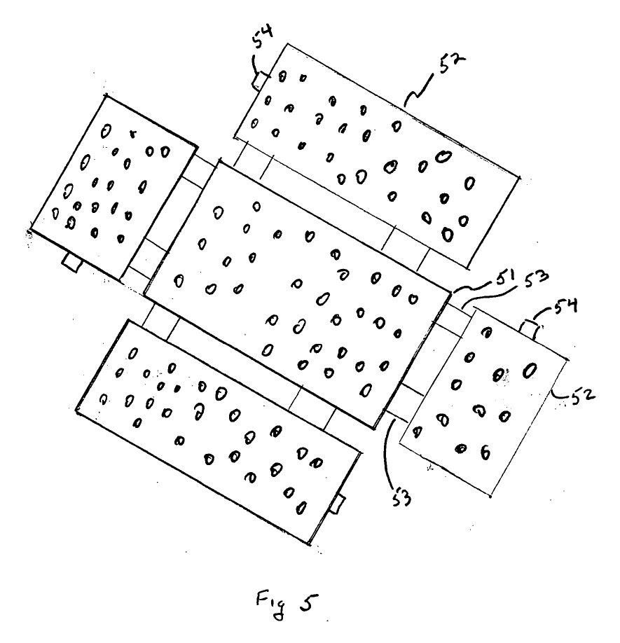

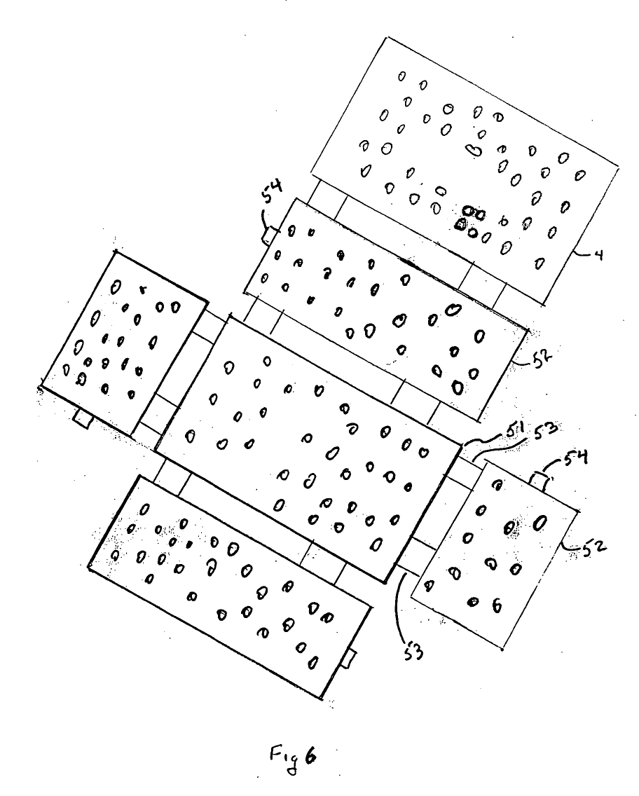

In an alternative embodiment (see FIG. 5), the outer case (1) is a hinged device having a bottom (51) and four side walls (52) where the hinged side walls are hinged to the bottom and as two adjacent walls are folded up, they interlock or lock together via a latching means (54). In using this type of construction, the open outer case (1) with the hingeably attached side walls not yet folded up is laid out on a reasonably flat surface, a liner (2) is placed thereover or a liner-wrapped inner tray is placed over the bottom (51). Two adjacent side walls (52) are raised and interlocked or latched together. The third and fourth side walls (52) are then raised in any sequence and interlocked or latched to the already standing sidewalls (52). The raised sidewalls (52) simultaneously raise the liner (2) that covered them (when the inner tray (3) was not previously wrapped) and once the side walls (52) are raised, the liner can be folded as discussed above. This embodiment, while being more time consuming, offers the advantage that the potential for ripping or tearing the liner (2) in the course of placing the liner (2) or the inner tray (3) into the outer case (1) is still further reduced, since the liner (2) and inner tray (3) do not have to slide past portions of the outer case (1) sidewalls during their placement into the outer case (1). In either situation, once the side walls (52) have been elevated and the inner tray (3) is wrapped in the liner (2), the four sidewalls (52) of the outer case (2) are secured by the outer case lid (4). In a preferred embodiment, the outer case lid (4) may be part of the hinged assembly and when stored may lay in flat sheets. In this preferred embodiment (FIG. 6), the outer case lid (4) is hingeably attached to a portion of one of the side walls 52, which edge is other than the edges that are attached to bottom (51) and the edges designed to latch to other sidewall (52) edges. In this way, the lid is always attached to the rest of the case that it fits and avoids misplacing the appropriate lid.

As a further alternative, the inner tray (3) may be simply a smaller sized outer case (without handles) that can fit within the larger outer case (1).

The present invention thus provides (1) a low cost means for providing a sterilization means, (2) a means for sterilization that is suitable for odd shaped or oversized items needing sterilization, and (3) improves sterilization maintenance by assuring fewer (if not completely eliminating) breaches in the wrappings used.

We claim:

1. A sterilization container system for sterilizing objects therein comprising
(a) an outer case having a bottom and four outer case side walls, said outer case bottom and said outer case four sidewalls defining an outer case interior;
(b) an outer case lid;
(c) a liner;
(d) an inner tray having an inner tray bottom and four inner tray side walls, said inner tray bottom and four inner tray side walls defining an inner tray interior; and
(e) optionally, an inner tray lid;
wherein in use, said inner tray carries one or more objects for sterilization in said inner tray interior, said inner tray optionally having said inner tray lid thereon, said inner tray along with said objects and optionally said inner tray lid being wrapped within said liner to form a wrapped inner tray, and said wrapped inner tray located in said outer case interior, with said outer case lid closing said interior of said outer case.

2. The system of claim 1 wherein at least one of said outer case bottom, said outer case side walls and said outer case lid contains perforations; and at least one of said inner tray bottom, said inner tray side walls, and if present, said optional inner tray lid contains perforations.

3. The system of claim 2 wherein each of said outer case bottom, said outer case side walls and said outer case lid, said inner tray bottom, said inner tray side walls, and if present, said optional inner tray lid contains perforations.

4. The system of claim 2 wherein said perforations in the inner tray are offset from said perforations in said outer case.

5. The system of claim 3 wherein said perforations in the inner tray are offset from said perforations in said outer case.

6. The system of claim 1 wherein said outer case is of a material selected from the group consisting of metal, metal alloy, anodized metal, anodized metal alloy, conductive plastic, polysulfone, polypropylene, liquid crystal polymer, and mixtures, blends, composites, and hybrids thereof.

7. The system of claim 6 wherein said outer case is constructed of a metal selected from the group consisting of stainless steel, aluminum, aluminum alloy, titanium, magnesium, mixtures thereof, blends thereof, and composites thereof; each of said metals optionally having an anodized layer thereon.

8. The system of claim 6 wherein said anodized metal and anodized metal alloy have an anodization layer not greater than 0.5 mils thick.

9. The system of claim 1 wherein said outer case lid is of a material selected from the group consisting of metal, metal alloy, anodized metal, anodized metal alloy, conductive plastic, liquid crystal polymer, and mixtures, blends, composites, and hybrids thereof.

10. The system of claim 9 wherein said outer case lid is constructed of a member selected from the group consisting of steel, stainless steel, aluminum, aluminum alloy, titanium, magnesium, mixtures thereof, blends thereof, and composites thereof; each of said metals optionally having an anodized layer thereon.

11. The system of claim 9 wherein said anodized metal and anodized metal alloy have an anodization layer not greater than 0.5 mils thick.

12. The system of claim 1 wherein said inner tray is of a material selected from the group consisting of metal, metal alloy, anodized metal, anodized metal alloy, conductive plastic, liquid crystal polymer, and mixtures, blends, composites, and hybrids thereof.

13. The system of claim 12 wherein said inner tray is constructed of a member selected from the group consisting of, stainless steel, aluminum, aluminum alloy, titanium, magnesium, mixtures thereof, blends thereof, and composites thereof; each of said metals optionally having an anodized layer thereon.

14. The system of claim 12 wherein said anodized metal and anodized metal alloy have an anodization layer not greater than 0.5 mils thick.

15. The system of claim 1 wherein said inner tray lid is of a material selected from the group consisting of metal, metal alloy, anodized metal, anodized metal alloy, conductive plastic, liquid crystal polymer, and mixtures, blends, composites, and hybrids thereof.

16. The system of claim 15 wherein said outer case lid is constructed of a member selected from the group consisting of, stainless steel, aluminum, aluminum alloy, titanium, magnesium, mixtures thereof, blends thereof, and composites thereof; each of said metals optionally having an anodized layer thereon.

17. The system of claim 16 wherein said anodized metal and anodized metal alloy have an anodization layer not greater than 0.5 mils thick.

18. The system of claim 1 wherein said liner is of a material selected from the group consisting of medical grade cellulosics, polypropylene, non-woven polyolefin, muslin, linen, synthetic wraps, and mixtures, blends, composites, and hybrids thereof.

19. The system of claim 18 wherein said synthetic wrap is selected from the group consisting of Gortex, Teflon, polyfoil compounds, tyvek, and mixtures, blends, composites, and hybrids thereof.

20. The system of claim 1 wherein said outer case bottom is hingeably connected to said outer case sidewalls.

21. The system of claim 1 wherein said outer case bottom has four edges and is hingeably connected to said four outer case sidewalls, being designated a first, second, third, and fourth outer case sidewalls, respectively, said first outer case sidewall being further hingeably connected to said outer case lid along an edge of said first outer case sidewall that is parallel to the edge of said first outer case sidewall that is hingeably connected to said outer case bottom.

22. The system of claim 1 wherein said liner further comprises a closure means.

23. The system of claim 22 wherein said closure means is Velcro.

24. A method of sterilizing objects in need thereof with the system of claim 1 comprising (a) placing said objects into said inner tray and optionally placing said inner tray lid on said inner tray; (b) wrapping said inner tray loaded with said objects and optionally closed with said optional inner tray lid to result in a wrapped inner tray; placing said wrapped inner tray into said outer case; placing said outer case lid on said outer case to form a sterilization unit, and subjecting said sterilization unit to a sterilization procedure.

25. A method of sterilizing objects in need thereof with the system of claim 1 comprising
(a) (i) placing said objects into said inner tray, optionally placing said inner tray lid on said inner tray;
(ii) placing said liner into said outer case interior; and
(iii) placing said inner tray with said objects to be disinfected onto said liner; or
(b) (i) placing said liner into said outer case interior;
(ii) placing said inner tray onto said liner; and
(iii) placing said objects into said inner tray, optionally placing said inner tray lid on said inner tray;
(c) wrapping said inner tray loaded with said objects and optionally closed with said optional inner tray lid to result in a wrapped inner tray;
(d) placing said outer case lid on said outer case to form a sterilization unit, and
(e) subjecting said sterilization unit to a sterilization procedure.

26. A method of sterilizing objects in need thereof with the system of claim 20 comprising
(a) placing said hingeably connected outer case bottom and side walls in an open position on a surface;
(b) placing said liner on said bottom and side walls;
(c) placing said inner tray on said liner,
(i) without said objects in said inner tray and then adding said objects into said inner tray or
(ii) with said objects having been previously placed into said inner tray
(d) optionally placing said inner tray lid on said inner tray; and
(e) in either sequence, (i) wrapping said inner tray in said liner and raising said outer case side walls and latching them into an upright position or (ii) first raising said outer case side walls to an upright position and latching them into place and then wrapping said inner tray with said liner;
(f) placing said outer case lid on said outer case to form a sterilization unit; and
(g) subjecting said sterilization unit to a sterilization procedure.
